SEQUENCE LISTING (21 sequences)

SEQ ID NO: 1 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 344 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 345 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 5 aa, synthetic construct
  REGION 1..5: note = Synthetic
GGGGS

SEQ ID NO: 10 - protein, 11 aa, synthetic construct
  REGION 1..11: note = Synthetic
GTELSVRAKPS

SEQ ID NO: 11 - protein, 5 aa, synthetic construct
  REGION 1..5: note = Synthetic peptides
SYVMH

SEQ ID NO: 12 - protein, 6 aa, synthetic construct
  REGION 1..6: note = Synthetic peptides
NPYNDG

SEQ ID NO: 13 - protein, 12 aa, synthetic construct
  REGION 1..12: note = Synthetic peptides
GTYYYGTRVFDY

SEQ ID NO: 14 - protein, 16 aa, synthetic construct
  REGION 1..16: note = Synthetic peptides
RSSKSLQNVNGNTYLY

SEQ ID NO: 15 - protein, 7 aa, synthetic construct
  REGION 1..7: note = Synthetic peptides
RMSNLNS

SEQ ID NO: 16 - protein, 9 aa, synthetic construct
  REGION 1..9: note = Synthetic peptides
MQHLEYPIT

SEQ ID NO: 17 - protein, 121 aa, synthetic construct
  REGION 1..121: note = Synthetic peptides
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 18 - protein, 112 aa, synthetic construct
  REGION 1..112: note = Synthetic peptides
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 19 - protein, 451 aa, synthetic construct
  REGION 1..451: note = Synthetic peptides
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 219 aa, synthetic construct
  REGION 1..219: note = Synthetic peptides
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 20 aa, synthetic construct
  VARIANT 6..20: note = May be absent
GGGGSGGGGSGGGGSGGGGS

(19) United States
(12) Patent Application Publication
(10) Pub. No.: US 2026/0166112 A1
(43) Pub. Date: Jun. 18, 2026

(54) COMBINATION THERAPY COMPRISING SIRP ALPHA FUSION PROTEIN AND ANTI-CD19 ANTIBODY FOR TREATMENT OF CANCER

(71) Applicant: Incyte Corporation, Wilmington, DE (US)

(72) Inventors: Ingmar BRUNS, Cambridge, MA (US); Victor Ruberio LINCHA, San Diego, CA (US); Sandra Jean MEECH, Groton, CT (US); Anita SCHEUBER, Boston, MA (US); Diane Dan WANG, San Diego, CA (US); Yibo WANG, San Diego, CA (US)

(21) Appl. No.: 18/872,419

(22) PCT Filed: Jun. 9, 2023
(86) PCT No.: PCT/US2023/068190

§ 371 Date: Dec. 6, 2024

Related U.S. Application Data
(60) Provisional application No. 63/351,117, filed on Jun. 10, 2022.
(60) Provisional application No. 63/494,500, filed on Apr. 6, 2023.

(51) Int. Cl. A61K 38/00; A61K 9/00

(52) U.S. Cl. CPC A61K 38/00; A61K 9/0019; A61K 9/0053

(57) ABSTRACT

Combination therapies comprising a SIRPaFc fusion protein and an anti-CD19 agent are provided. The combination therapies can optionally further include an E3 ubiquitin ligase modulator.

### REFERENCE TO AN ELECTRONIC SEQUENCE LISTING

This application contains a Sequence Listing that has been submitted electronically as an XML file named P113970010WO00-SEQ-LJG.xml. The XML file, created on Aug. 21, 2025, is 26,830 bytes in size. The material in the XML file is hereby incorporated by reference in its entirety.

### BACKGROUND

Cancer cells are targeted for destruction by antibodies that bind to cancer cell antigens, and through recruitment and activation of macrophages by way of Fc receptor binding to the Fc portion of that antibody. Binding between CD47 on cancer cells and SIRP alpha (“SIRPa”) on macrophages transmits a “don't eat me” signal that enables many tumour cells to escape destruction by macrophages. It has been shown that inhibition of the CD47/SIRPa interaction (CD47 blockade) will allow macrophages to “see” and destroy the target CD47+ cancer cell. The use of SIRPa to treat cancer by CD47 blockade is described in WO 2010/130053, incorporated herein by reference.

International Patent Application Publication No. WO 2014/094122, incorporated by reference in its entirety, describes a protein drug that inhibits the interaction between CD47 and SIRPa. This CD47 blockade drug is a form of human SIRPa that incorporates a particular region of its extracellular domain linked with a particularly useful form of an IgG-based Fc region. In this form, the SIRPaFc drug shows dramatic effects on the viability of cancer cells that present with a CD47+ phenotype. The effect is seen particularly on acute myelogenous leukemia (AML) cells, and on many other types of cancer.

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signaling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signaling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR208 and XmAb®5574) is a humanized monoclonal antibody that targets the antigen CD19, a transmembrane protein involved in B-cell receptor signaling. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. Tafasitamab received accelerated approval from the US Food and Drug Administration (FDA) in July 2020 for use in combination with lenalidomide to treat adults with R/R DLBCL. In August 2021, the European Commission granted conditional marketing authorization for tafasitamab in combination with lenalidomide, followed by tafasitamab monotherapy, for the treatment of adult patients with relapsed or refractory diffuse large B-cell lymphoma (DLBCL) who are not eligible for autologous stem cell transplantation (ASCT). Despite recent discoveries and developments of several anti-cancer agents, due to poor prognosis for many types of cancers including CD19-expressing tumors, there is still a need for an improved method or therapeutic approach for treating such types of cancers.

Accordingly, improved dosing regimens and treatment methods using SIRPaFc agents and CD19-targeting agents are needed.

### SUMMARY

Provided herein are therapies combining SIRPaFc fusion proteins and CD19-targeting agents. Dosing regimens and methods provided herein include both a SIRPaFc fusion protein and an anti-CD19 antibody. The combination therapies may further comprise an E3 ubiquitin ligase modulator.

In some embodiments, provided herein is a method of treating a cancer in a patient, comprising administering a combination of a SIRPaFc fusion protein and an anti-CD19 antibody to the patient.

In some embodiments, provided herein is a method of treating a cancer in a patient, the method comprising administering a combination therapy of SIRPaFc fusion protein, an anti-CD19 antibody, and an E3 ubiquitin ligase modulator to the patient for at least a first cycle, a second cycle, a third cycle, and a fourth cycle, wherein each cycle is 28 days, wherein in the first cycle the SIRPaFc fusion protein is administered on days 1, 8, 15, and 22, the anti-CD19 antibody is administered on days 1, 4, 8, 15, and 22, and the E3 ubiquitin ligase modulator is administered on days 1-21, wherein in the second cycle the SIRPaFc fusion protein is administered on days 1, 8, 15, and 22, the anti-CD19 antibody is administered on days 1, 8, 15, and 22, and the E3 ubiquitin ligase modulator is administered on days 1-21, wherein in the third cycle the SIRPaFc fusion protein is administered on days 1, 8, 15, and 22, the anti-CD19 antibody is administered on days 1, 8, 15, and 22, and the E3 ubiquitin ligase modulator is administered on days 1-21, and wherein in the fourth cycle the SIRPaFc fusion protein is administered on days 1 and 15, the anti-CD19 antibody is administered on days 1 and 15, and the E3 ubiquitin ligase modulator is administered on days 1-21.

In some embodiments, provided herein is a method of treating a cancer in a patient, the method comprising administering a combination therapy of SIRPaFc fusion protein, an anti-CD19 antibody, and an E3 ubiquitin ligase modulator to the patient for at least a first cycle, a second cycle, a third cycle, and a fourth cycle, wherein each cycle is 28 days, wherein in the first cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1, 4, 8, 15, and 22 at a dose of 12 mg/kg, and the E3 ubiquitin ligase modulator is administered on days 1-21 at a dose of 25 mg, wherein in the second cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg the anti-CD19 antibody is administered on days 1, 8, 15, and 22 at a dose of 12 mg/kg, and the E3 ubiquitin ligase modulator is administered on days 1-21 at a dose of 25 mg, wherein in the third cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1, 8, 15, and 22 at a dose of 12 mg/kg, and the E3 ubiquitin ligase modulator is administered on days 1-21 at a dose of 25 mg, wherein in the fourth cycle the SIRPaFc fusion protein is administered on days 1 and 15 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg the anti-CD19 antibody is administered on days 1 and 15 at a dose of 12 mg/kg, and the E3 ubiquitin ligase modulator is administered on days 1-21 at a dose of 25 mg, and wherein the SIRPaFc fusion protein is TTI-622, the anti-CD19 antibody is tafasitamab, and the E3 ubiquitin ligase modulator is lenalidomide.

### BRIEF DESCRIPTION OF THE DRAWINGS

FIG. 1 depicts an exemplary schema for a combination therapy combining TTI-622 (SIRPaFc fusion protein), the tafasitamab (anti-CD19 antibody), and lenalidomide (E3 ubiquitin ligase modulator).

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. A method of treating a cancer in a patient, comprising administering a combination of a SIRPaFc fusion protein and an anti-CD19 antibody to the patient.

E2. The method of E1, wherein the method further comprises administering an E3 ubiquitin ligase modulator.

E3. The method of any one of E1-E2, wherein the SIRPaFc fusion protein is administered Q1W or Q2W.

E4. The method of any one of E1-E3, wherein the anti-CD19 antibody is administered Q1W or Q2W.

E5. The method of any one of E1-E4, wherein the E3 ubiquitin ligase modulator is administered daily.

E6. A method of treating a cancer in a patient, the method comprises administering a combination therapy of SIRPaFc fusion protein, an anti-CD19 antibody, and an E3 ubiquitin ligase modulator to the patient for at least a first cycle, a second cycle, a third cycle, and a fourth cycle,
wherein each cycle is 28 days,
wherein in the first cycle the SIRPaFc fusion protein is administered on days 1, 8, 15, and 22, the anti-CD19 antibody is administered on days 1, 4, 8, 15, and 22, and the E3 ubiquitin ligase modulator is administered on days 1-21,
wherein in the second cycle the SIRPaFc fusion protein is administered on days 1, 8, 15, and 22, the anti-CD19 antibody is administered on days 1, 8, 15, and 22, and the E3 ubiquitin ligase modulator is administered on days 1-21,
wherein in the third cycle the SIRPaFc fusion protein is administered on days 1, 8, 15, and 22, the anti-CD19 antibody is administered on days 1, 8, 15, and 22, and the E3 ubiquitin ligase modulator is administered on days 1-21, and
wherein in the fourth cycle the SIRPaFc fusion protein is administered on days 1 and 15, the anti-CD19 antibody is administered on days 1 and 15, and the E3 ubiquitin ligase modulator is administered on days 1-21.

E7. The method of E6, wherein the method is continued for at least a fifth cycle and up to a twelfth cycle, and in the fifth to the twelfth cycle the SIRPaFc fusion protein is administered on days 1 and 15, the anti-CD19 antibody is administered on days 1 and 15, and the E3 ubiquitin ligase modulator is administered on days 1-21.

E8. The method of E7, wherein the method is continued for at least a thirteenth cycle, wherein in the thirteenth cycle the SIRPaFc fusion protein is administered on days 1 and 15, the anti-CD19 antibody is administered on days 1 and 15, and no E3 ubiquitin ligase modulator is administered.

E9. The method of any one of E1-E8, wherein the SIRPaFc fusion protein is administered at a dose of 4 mg/kg, 10 mg/kg, or 18 mg/kg, or a fixed dose of 300 mg, 600 mg, 750 mg, 1200 mg, 1350 mg, 1500 mg, 1800 mg, 2100 mg, or 2400 mg.

E10. The method of any one of E1-E9, wherein the anti-CD19 antibody is administered at a dose of 12 mg/kg.

E11. The method of any one of E1-E10, wherein the E3 ubiquitin ligase modulator is administered at a dose of 25 mg.

E12. A method of treating a cancer in a patient, the method comprises administering a combination therapy of SIRPaFc fusion protein, an anti-CD19 antibody, and an E3 ubiquitin ligase modulator to the patient for at least a first cycle, a second cycle, a third cycle, and a fourth cycle,
wherein each cycle is 28 days,
wherein in the first cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1, 4, 8, 15, and 22 at a dose of 12 mg/kg, and the E3 ubiquitin ligase modulator is administered on days 1-21 at a dose of 25 mg,
wherein in the second cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1, 8, 15, and 22 at a dose of 12 mg/kg, and the E3 ubiquitin ligase modulator is administered on days 1-21 at a dose of 25 mg,
wherein in the third cycle the SIRPaFc fusion protein is administered on days 1, 8, 15, and 22 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg the anti-CD19 antibody is administered on days 1, 8, 15, and 22 at a dose of 12 mg/kg, and the E3 ubiquitin ligase modulator is administered on days 1-21 at a dose of 25 mg, and
wherein in the fourth cycle the SIRPaFc fusion protein is administered on days 1 and 15 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and the E3 ubiquitin ligase modulator is administered on days 1-21 at a dose of 25 mg.

E13. The method of E12, wherein the method is continued for at least a fifth cycle and up to a twelfth cycle, and in the fifth to the twelfth cycle the SIRPaFc fusion protein is administered on days 1 and 15 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and the E3 ubiquitin ligase modulator is administered on days 1-21 at a dose of 25 mg.

E14. The method of E13, wherein the method is continued for at least a thirteenth cycle, wherein in the thirteenth cycle the SIRPaFc fusion protein is administered on days 1 and 15 at a dose of 4 mg/kg, 10 mg/kg, 18 mg/kg, or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and no E3 ubiquitin ligase modulator is administered.

E15. The method of any one of E1-E14, wherein the SIRPaFc fusion protein comprises a SIRPa polypeptide comprising the amino acid sequence of SEQ ID NO: 1.

E16. The method of any one of E1-E15, wherein the SIRPaFc fusion protein comprises a SIRPa polypeptide comprising the amino acid sequence of SEQ ID NO: 2.

E17. The method of any one of E1-E16, wherein the SIRPaFc fusion protein comprises the amino acid sequence of SEQ ID NO: 7.

E18. The method of any one of E1-E16, wherein the SIRPaFc fusion protein comprises the amino acid sequence of SEQ ID NO: 8.

E19. The method of any one of E1-E15, wherein the SIRPaFc fusion protein comprises a SIRPa polypeptide comprising the amino acid sequence of SEQ ID NO: 1 or a variant thereof having one, two, three, four, or five amino acid substitutions as compared the sequence of SEQ ID NO: 1.

E20. The method of any one of E1-E19, wherein the anti-CD19 antibody comprises a heavy chain variable region comprising an HCDR1 region comprising the sequence SYVMH (SEQ ID NO: 11), an HCDR2 region comprising the sequence NPYNDG (SEQ ID NO: 12), and an HCDR3 region comprising the sequence GTYYYGTRVFDY (SEQ ID NO: 13) and a light chain variable region comprising the sequence LCDR1 region comprising the sequence RSSKSLQNVNGNTYLY (SEQ ID NO: 14), an LCDR2 region comprising the sequence RMSNLNS (SEQ ID NO: 15), and an LCDR3 region comprising the sequence MQHLEYPIT (SEQ ID NO: 16).

E21. The method of any one of E1-E20, wherein the anti-CD19 antibody comprises a heavy chain variable region of

(SEQ ID NO: 17)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSS
and
a light chain variable region of
(SEQ ID NO: 18)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIK.

E22. The method of any one of E1-E21, wherein the anti-CD19 antibody comprises an Fc domain comprising an amino acid substitution at position S239 and/or I332, wherein the numbering is according to the EU index as in Kabat.

E23. The method of E22, wherein the anti-CD19 antibody comprises an Fc domain comprising an S239D amino acid substitution and an I332E amino acid substitution, wherein the numbering is according to the EU index as in Kabat.

E24. The method of any one of E1-E23, wherein the anti-CD19 antibody comprises a heavy chain region of

(SEQ ID NO: 19)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPDVF
LFPPKPKDTLMISRTPEVTCVVVDVSHEDPEVQFNWYVDGVEVHNAKTK
PREEQFNSTFRVVSVLTVVHQDWLNGKEYKCKVSNKALPAPEEKTISKT
KGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYT
QKSLSLSPGK
and
a light chain region of
(SEQ ID NO: 20)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

E25. The method of any one of E1-E24, wherein the E3 ubiquitin ligase modulator comprises lenalidomide, pomalidomide, or thalidomide.

E26. The method of any one of E1-E25, wherein the E3 ubiquitin ligase modulator is lenalidomide.

E27. The method of any one of E1-E26, wherein the E3 ubiquitin ligase modulator is administered orally.

E28. The method of any one of E1-E27, wherein the SIRPaFc fusion protein and the anti-CD19 antibody are administered intravenously or subcutaneously.

E29. The method of any one of E1-E28, wherein the cancer is a CD19 positive cancer.

E30. The method of any one of E1-E29, wherein the cancer is a CD47 positive cancer.

E31. The method of any one of E1-E30, wherein the cancer is a hematological malignancy or a solid tumor cancer.

E32. The method of any one of E1-E31, wherein the cancer is a lymphoma or a leukemia.

E33. The method of any one of E1-E32, wherein the cancer is chronic lymphocytic leukemia, acute lymphoblastic leukemia, or a non-Hodgkin's lymphoma.

E34. The method of any one of E1-E33, wherein the cancer is a non-Hodgkin's lymphoma selected from the group consisting of follicular lymphoma (FL), small lymphocytic lymphoma, mucosa-associated lymphoid tissue lymphoma, marginal zone lymphoma (MZL), mantle cell lymphoma (MCL), diffuse large B-cell lymphoma (DLBCL), and Burkitt lymphoma.

E35. A SIRPaFc fusion protein or an anti-CD19 antibody for use to treat a patient according to the method of any one of E1-E34.

E36. Use of a SIRPaFc fusion protein or an anti-CD19 antibody in the manufacture of a medicament for use to treat a patient according to the method of any one of E1-E34. E37. A kit comprising a SIRPaFc fusion protein or an anti-CD19 antibody and instructions for use according to the method of any one of claims 1-34, and optionally further comprising one or more additional therapeutic agents according to the method of any one of E1-E34.

Without being bound by theory, in combination methods provided herein involving a SIRPaFc fusion protein (e.g. TTI-622), an anti-CD19 antibody (e.g. tafasitamab), and an E3 ubiquitin ligase modulator (e.g. lenalidomide), it is believed that one or more of the following anti-cancer effects may occur in view of the combination treatment: 1) increased antibody-dependent cellular phagocytosis (ADCP): tafasitamab and lenalidomide increase the “eat” signal, whereas TTI-622 blocks the “don't eat me” signal on tumor cells; 2) increased antibody-dependent cellular cytotoxicity (ADCC): lenalidomide lowers the threshold of NK cell activation, thereby facilitating tafasitamab mediated ADCC; 3) directed killing: lenalidomide has direct tumor killing activity. These activities may occur in various cancer types, such as DLBCL.

In some embodiments, the present disclosure relates to the use of a SIRPaFc fusion protein in combination with an anti-CD19 antibody for the treatment of a cancer in a patient.

In some embodiments, the present disclosure relates to a composition comprising a SIRPaFc fusion protein for use in treating a cancer in a patient, wherein the patient is also treated with an anti-CD19 antibody. In some aspects, the disclosure relates to a composition comprising a SIRPaFc fusion protein for use in treating a disorder which is cancer in a patient, wherein the SIRPaFc fusion protein is in combination with the anti-CD19 antibody. In some embodiments, the SIRPaFc fusion protein can be administered simultaneously or sequentially with the anti-CD19 antibody.

In some embodiments, the present disclosure relates to methods of treating a cancer comprising administering to a patient in need of such treatment, a therapeutically effective amount of a combination of a SIRPaFc fusion protein and an anti-CD19 antibody.

In some embodiments, the present disclosure relates to a method of treating a cancer by administering to a patient a combination of a SIRPaFc fusion protein or pharmaceutically acceptable salt thereof, and an anti-CD19 antibody.

In another aspect, the present disclosure relates to the use of a SIRPaFc fusion protein in combination with an anti-CD19 antibody for the treatment of a cancer.

In some embodiments, the methods of treating a cancer, as described herein, can include a combination of a SIRPaFc fusion protein and an anti-CD19 antibody, and one or more additional therapeutic agents. In some embodiments, the one or more additional therapeutic agents can be chemotherapeutic agents. In some embodiments, the one or more additional therapeutic agents can include, but are not limited to lenalidomide, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof.

In some embodiments, the methods of treating a cancer, as described herein, can include a combination of a SIRPaFc fusion protein, an anti-CD19 antibody and lenalidomide.

In some embodiments, the methods of treating a cancer, as described herein, can include a combination of a SIRPaFc fusion protein and an anti-CD19 antibody, wherein said anti-CD19 antibody is tafasitamab and wherein tafasitamab is administered in a 12 mg/kg body weight dose.

In some embodiments, the methods of treating a cancer, as described herein, can include a combination of a SIRPaFc fusion protein and an anti-CD19 antibody, wherein the SIRPaFc fusion protein is TTI-622 and wherein TTI-622 is administered in a 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg body weight dose on day 1, day 8, day 15, and day 22 of a first, second, and third 28 day cycle and on day 1 and day 15 of following 28 day cycles, wherein said anti-CD19 antibody is tafasitamab and wherein tafasitamab is administered in a 12 mg/kg body weight dose on day 1, day 4, day 8, day 15 and day 22 of the first 28-day cycle and on day 1, day 8, day 15 and day 22 of the second and third 28-day cycle and on day 1 and 15 of one or more the following cycles.

In some embodiments, the methods of treating a cancer, as described herein, can include a combination of a SIRPaFc fusion protein, an anti-CD19 antibody and lenalidomide, wherein the SIRPaFc fusion protein is TTI-622 and wherein TTI-622 is administered in a 4 mg/kg, 10 mg/kg, or 18 mg/kg body weight dose or a fixed dose of 300 mg, 600 mg, 750 mg, 1200 mg, 1350 mg, 1500 mg, 1800 mg, 2100 mg, or 2400 mg, wherein said anti-CD19 antibody is tafasitamab and wherein tafasitamab is administered in a 12 mg/kg body weight dose wherein lenalidomide is administered in a 25 mg dose.

In some embodiments, the methods of treating a cancer, as described herein, can include a combination of a SIRPaFc fusion protein, an anti-CD19 antibody and lenalidomide, wherein the SIRPaFc fusion protein is TTI-622 and wherein TTI-622 is administered in a 4 mg/kg, 10 mg/kg, or 18 mg/kg body weight dose or a fixed dose of 300 mg, 600 mg, 750 mg, 1200 mg, 1350 mg, 1500 mg, 1800 mg, 2100 mg, or 2400 mg, wherein said anti-CD19 antibody is tafasitamab and wherein tafasitamab is administered in a 12 mg/kg body weight dose wherein lenalidomide is administered in a 25 mg dose on days 1 to 21 of a 28-day cycle.

In some embodiments, the methods of treating a cancer, as described herein, can include a combination of a SIRPaFc fusion protein, an anti-CD19 antibody and lenalidomide, wherein the SIRPaFc fusion protein is TTI-622 and wherein TTI-622 is administered in a 4 mg/kg, 10 mg/kg, or 18 mg/kg body weight dose or a fixed dose of 300 mg, 600 mg, 750 mg, 1200 mg, 1350 mg, 1500 mg, 1800 mg, 2100 mg, or 2400 mg on day 1, day 8, day 15, and day 22 of a first, second, and third 28 day cycle and on day 1 and day 15 of one or more following 28 day cycles, wherein said anti-CD19 antibody is tafasitamab and wherein tafasitamab is administered in a 12 mg/kg body weight dose on day 1, day 4, day 8, day 15 and day 22 of the first 28-day cycle and on day 1, day 8, day 15 and day 22 of the second and third 28-day cycle and on day 1 and 15 of one or more the following and wherein lenalidomide is administered in a 25 mg dose on days 1 to 21 of the 28-day cycle.

In some embodiments, the cancer is a CD19-positive cancer.

In some embodiments, the cancer is a hematological cancer. Non-limiting examples of hematologic cancers include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's lymphoma (HL); non-Hodgkin's lymphoma (NHL), including B-cell lymphoma, T-cell lymphoma, follicular lymphoma (FL), marginal zone lymphoma (MZL), mantle cell lymphoma (MCL), diffuse large B-cell lymphoma (DLBCL), and Burkitt lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes. In some embodiments, the cancer is chronic lymphocytic leukemia (CLL), Hodgkin's lymphoma, or non-Hodgkin's lymphoma including follicular lymphoma (FL), marginal zone lymphoma (MZL), mantle cell lymphoma (MCL), diffuse large B-cell lymphoma (DLBCL) and Burkitt lymphoma.

In some embodiments, the cancer is chronic lymphocytic leukemia. In some embodiments, the cancer is CD19-positive chronic lymphocytic leukemia.

In some embodiments, the cancer is a non-Hodgkin's lymphoma, including follicular lymphoma (FL), marginal zone lymphoma (MZL), mantle cell lymphoma (MCL), Diffuse large B-cell lymphoma (DLBCL), and Burkitt lymphoma. In some embodiments, the cancer is a B-cell lymphoma. In some embodiments, the cancer is a CD19-positive non-Hodgkin's lymphoma. In some embodiments, the cancer is a CD19-positive aggressive non-Hodgkin lymphoma. In some embodiments, the cancer is a CD19-positive indolent non-Hodgkin lymphoma. In some embodiments, the cancer is relapsed or refractory non-Hodgkin lymphoma. In some embodiments, the cancer is relapsed or refractory CD19-positive aggressive non-Hodgkin lymphoma. In some embodiments, the cancer is relapsed or refractory CD19-positive indolent non-Hodgkin lymphoma.

In some embodiments, the cancer is relapsed. In some embodiments, relapsed cancer is cancer which has returned after a period of time in which no cancer could be detected.

In some embodiments, the cancer is refractory. In some embodiments, refractory cancer does not respond to cancer treatment; it is also known as resistant cancer. In some embodiments, the cancer is resistant to rituximab. In some embodiments, the cancer does not respond to the treatment of rituximab. In some embodiments, the cancer is rituximab-resistant recurrent cancer. In some embodiments, the patient has become refractory to a rituximab-containing regimen. In some embodiments, the tumor is unresectable. In some embodiments, an unresectable tumor is unable to be removed by surgery. In some embodiments, the cancer has not been previously treated. In some embodiments, the cancer is locally advanced. In some embodiments, “locally advanced” refers to cancer that is somewhat extensive but still confined to one area. In some instances, “locally advanced” may refer to a small tumor that hasn't spread but has invaded nearby organs or tissues that make it difficult to remove with surgery alone. In some embodiments, the cancer is metastatic. In some embodiments, metastatic cancer is a cancer that has spread from the part of the body where it started (the primary site) to one or more other parts of the body.

In some embodiments, the patient has relapsed or refractory CD19-positive non-Hodgkin lymphoma. In some embodiments, the patient has both CD19-positive non-Hodgkin lymphoma and relapsed or refractory non-Hodgkin lymphoma.

In some embodiments, the patient has relapsed or refractory CD19-positive aggressive non-Hodgkin lymphoma. In some embodiments, the patient has relapsed or refractory CD19-positive aggressive non-Hodgkin lymphoma and has progressed on at least one prior treatment regimen.

In some embodiments, the patient has relapsed or refractory CD19-positive indolent non-Hodgkin lymphoma. In some embodiments, the patient has relapsed or refractory CD19-positive indolent non-Hodgkin lymphoma and has progressed on at least two prior treatment regimens. In some embodiments, the patient has relapsed or refractory CD19-positive indolent non-Hodgkin lymphoma and is refractory to any anti-CD20 monoclonal antibody or any anti-CD19 monoclonal antibody. In some embodiments, the patient has relapsed or refractory CD19-positive indolent non-Hodgkin lymphoma and has progressed on at least two prior treatment regimens and is refractory to any anti-CD20 monoclonal antibody.

In some embodiments, the present disclosure relates to a medicament for use in treating a cancer in a patient in need of such treatment. The medicament comprises a SIRPaFc fusion protein and an anti-CD19 antibody, and is in single dosage form or in separate dosage forms.

In some embodiments, the medicaments, as described herein, can include a combination of a SIRPaFc fusion protein, an anti-CD19 antibody, and optionally one or more additional therapeutic agents.

In some embodiments, the present disclosure relates to the use of a SIRPaFc fusion protein in the manufacture of a medicament for treating a cancer, wherein the SIRPaFc fusion protein is administered with an anti-CD19 antibody, and wherein the medicament is in single dosage form or in separate dosage forms. In some embodiments, the SIRPaFc fusion protein is administered with an anti-CD19 antibody and one or more additional therapeutic agents.

In some embodiments, the present disclosure relates to the use of a SIRPaFc fusion protein for the manufacture of a medicament in treating a cancer in a patient, wherein the patient is also treated with an anti-CD19 antibody, and optionally one or more additional therapeutic agents. In some embodiments, the SIRPaFc fusion protein may be administered simultaneously or sequentially with the anti-CD19 antibody.

In another aspect, the present disclosure relates to the use of a SIRPaFc fusion protein, in combination with an anti-CD19 antibody in the manufacture of a medicament for use in treating a cancer. In some embodiments, the present disclosure relates to the use of a SIRPaFc fusion protein, in combination with an anti-CD19 antibody, and optionally one or more additional therapeutic agents in the manufacture of a medicament for use in treating a cancer.

In another aspect, the present disclosure relates to the use of a SIRPaFc fusion protein in the manufacture of a medicament for treating a cancer, wherein a SIRPaFc fusion protein is administered with an anti-CD19 antibody, and optionally one or more additional therapeutic agents.

In some embodiments, the one or more additional therapeutic agents can be chemotherapeutic agents. In some embodiments, the one or more additional therapeutic agents can include, but are not limited to, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof.

In one aspect, the present disclosure relates to an anti-CD19 antibody for use in the treatment of a cancer, wherein said anti-CD19 antibody is administered in combination with a SIRPaFc fusion protein to a subject in need of such treatment.

In one aspect, the present disclosure relates to an anti-CD19 antibody for use in the treatment of a cancer, wherein said anti-CD19 antibody is administered in combination with a SIRPaFc fusion protein.

In some embodiments, a SIRPaFc fusion protein may be administered in combination with the anti-CD19 antibody, and optionally one or more additional therapeutic agents, in a single dosage form or as a separate dosage forms. In some embodiments, when administered as a separate dosage form, the anti-CD19 antibody may be administered prior to, at the same time as, or following administration of the SIRPaFc fusion protein. In some embodiments, when administered as a separate dosage form, one or more doses of the SIRPaFc fusion protein, may be administered prior to the anti-CD19 antibody. In some embodiments, the anti-CD19 antibody is administered prior to the administration of the SIRPaFc fusion protein. As used herein, the administration in “combination” of a SIRPaFc fusion protein, an anti-CD19 antibody, and optionally one or more additional therapeutic agents refers not only to simultaneous or sequential administration of the agents, but also to the administration of the agents during a single treatment cycle, as understood by one skilled in the art. When a SIRPaFc fusion protein is administered in combination with the anti-CD19 antibody, and optionally one or more additional therapeutic agents, a therapeutically effective amount of the combination is administered.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of SIRPaFc fusion protein or anti-CD19 antibody) means that the parameter may vary by as much as 10% below or above the stated numerical value for that parameter. For example, a dose of about 5 mg means 5%±10%, i.e., it may vary between 4.5 mg and 5.5 mg.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is cancer.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and (3) ameliorating the disease; for example, ameliorating a disease, condition or disorder in an individual that is experiencing or displaying the pathology or symptomatology of the disease, condition or disorder (i.e., reversing the pathology or symptomatology).

### SIRPaFc Fusion Proteins

The present invention provides combination therapies that include SIRPalpha-Fc (“SIRPaFc”) fusion proteins.

Dosing regimens and methods provided herein use a CD47-binding and blocking form of SIRPa, as a CD47 blockade drug or blocking agent. An agent or drug that has CD47 blockade activity is an agent that interferes with and dampens signal transmission that results when CD47 interacts with macrophage-presented SIRPa. CD47-binding forms of human SIRPa are the preferred CD47 blockade drugs for use in the regimens and methods provided herein.

These drugs are based on the extracellular region of human SIRPa. They comprise at least a region of the extracellular region sufficient to confer effective CD47 binding affinity and specificity. So-called “soluble” forms of SIRPa, lacking the membrane anchoring component, are described in the literature and include those referenced in WO 2010/070047 (Novartis), WO2013/109752 (Stanford), and WO2014/094122 (Trillium), each incorporated by reference in its entirety.

In a preferred embodiment, the soluble form of SIRPa is an Fc fusion. More particularly, the drug suitably comprises the human SIRPa protein, in a form fused directly, or indirectly, with an antibody constant region, or Fc (fragment crystallisable). Unless otherwise stated, the term “human SIRPa” as used herein refers to a wild type, endogenous, mature form of human SIRPa. In humans, the SIRPa protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPa constitute about 80% of the forms of SIRPa present in humans, and both are embraced herein by the term “human SIRPa”. Also embraced by the term “human SIRPa” are the minor forms thereof that are endogenous to humans and have the same property of triggering signal transduction through CD47 upon binding thereto. The present invention is directed most particularly to the drug combinations that include the human SIRPa variant 2 form, or V2.

In the dosing regimens and methods provided herein, useful SIRPaFc fusion proteins comprise one of the three so-called immunoglobulin (Ig) domains that lie within the extracellular region of human SIRPa. More particularly, the present SIRPaFc proteins incorporate residues 32-137 of human SIRPa (a 106-mer), which constitute and define the IgV domain of the V2 form according to current nomenclature. This SIRPa sequence, shown below, is referenced herein as SEQ ID NO: 1.

[SEQ ID NO: 1]
EELQVIQPDKSVSVAAGESAILHCTVTSLIPVGPIQWFRGAGPARELIY
NQKEGHFPRVTTVSESTKRENMDFSISISNITPADAGTYYCVKFRKGSP
DTEFKSGA

In some embodiments, SIRPaFc fusion proteins incorporate the IgV domain as defined by SEQ ID NO: 1, and additional, flanking residues contiguous within the SIRPa sequence. This form of the IgV domain, represented by residues 31-148 of the V2 form of human SIRPa, is a 118-mer having SEQ ID NO: 2 shown below:

[SEQ ID NO: 2]
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPS

The present SIRPa fusion proteins can also incorporate an Fc region having effector function. Fc refers to “fragment crystallisable” and represents the constant region of an antibody comprised principally of the heavy chain constant region and components within the hinge region. Suitable Fc components include those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In some embodiments, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 3:

[SEQ ID NO: 3]
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

Thus, in some embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In some embodiments, the Fc region is based on the amino acid sequence of a human IgG4 set out as P01861 in UniProtKB/Swiss-Prot, residues 99-327, and has the amino acid sequence shown below and referenced herein as SEQ ID NO: 4:

[SEQ ID NO: 4]
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In some embodiments, the Fc region incorporates one or more alterations, usually not more than about 10, e.g., up to 1, 2, 3, 4, 5 or 6 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. Pat. No. 62/777,375, and many others. Particularly useful are those alterations that enhance Fc properties while remaining silent with respect to conformation, e.g., retaining Fc receptor binding. In another embodiment, the Fc region is modified to increase its biological half-life. Various approaches are possible. For example, one or more of the following mutations can be introduced; T252L, T254S, T256F, as described in U.S. Pat. No. 6,277,375.

In a specific embodiment, and in the case where the Fc component is an IgG4 Fc, the Fc incorporates at least the S²²⁸P mutation, and has the amino acid sequence set out below and referenced herein as SEQ ID NO: 5:

[SEQ ID NO: 5]
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The CD47 blockade drug used in the regimens and method provided herein is thus preferably a SIRPa fusion protein useful to inhibit the binding of human SIRPa and human CD47, thereby to inhibit or reduce transmission of the signal mediated via SIRPa-bound CD47, the fusion protein comprising a human SIRPa component and, fused therewith, an Fc component, wherein the SIRPa component comprises or consists of a single IgV domain of human SIRPa V2 and the Fc component is the constant region of a human IgG having effector function.

In one embodiment, the fusion protein comprises a SIRPa component consisting at least of residues 32-137 of the V2 form of wild type human SIRPa, i.e., SEQ ID NO: 1. In a preferred embodiment, the SIRPa component consists of residues 31-148 of the V2 form of human SIRPa, i.e., SEQ ID NO: 2. In another embodiment, the Fc component is the Fc component of the human IgG1 designated P01857, and in a specific embodiment has the amino acid sequence that incorporates the lower hinge-CH2-CH3 region thereof i.e., SEQ ID NO: 3.

In some embodiments, the SIRPaFc fusion protein is provided and used in a secreted dimeric fusion form, wherein the fusion protein incorporates a SIRPa component having SEQ ID NO: 1 and preferably SEQ ID NO: 2 and, fused therewith, an Fc region having effector function and having SEQ ID NO: 3. When the SIRPa component is SEQ ID NO: 1, this fusion protein comprises SEQ ID NO: 6, shown below:

[SEQ ID NO: 6]
EELQVIQPDKSVSVAAGESAILHCTVTSLIPVGPIQWFRGAGPARELIY
NQKEGHFPRVTTVSESTKRENMDFSISISNITPADAGTYYCVKFRKGSP
DTEFKSGAGTELSVRAKPSDKTHTCPPCPAFELLGGPSVFLFPPKPKDT
LMISRTPEVTCVVVDVSHEDPEVKFNWYVDGVEVHNAKTKPREEGYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K

When the SIRPa component is SEQ ID NO: 2, this fusion protein comprises SEQ ID NO: 7, shown below:

[SEQ ID NO: 7]
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

The SIRPaFc fusion protein of SEQ ID NO: 7 is also known as TTI-621.

In alternative embodiments, the Fc component of the fusion protein is based on an IgG4, and preferably an IgG4 that incorporates the S²²⁸P mutation. In the case where the fusion protein incorporates the preferred SIRPa IgV domain of SEQ ID NO: 2, the resulting IgG4-based SIRPa-Fc protein has SEQ ID NO: 8, shown below:

[SEQ ID NO: 8]
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

The SIRPaFc fusion protein of SEQ ID NO: 8 is also known as TTI-622.

In one embodiment of a dosing regimen or method provided herein, a SIRPaFc fusion protein comprises, as the SIRPa component of the fusion protein, a sequence that comprises SEQ ID NO: 2. In one embodiment, the SIRPaFc fusion protein comprises the polypeptide of SEQ ID NO: 7 or SEQ ID NO: 8.

The SIRPa sequence incorporated within the SIRPaFc fusion protein can be varied, as described in the literature. This can eliminate glycosylation sites in the protein, such as at position 89 and elsewhere. Other, useful substitutions within SIRPa include one or more of the following: L4V/I, V61/L, A21V, V27I/L, 131T/S/F, E47V/L, K53R, E54Q, H56P/R, S66T/G, K68R, V92I, F94V/L, V63I, and/or F103V.

In the SIRPaFc fusion protein, the SIRPa component and the Fc component are fused, either directly or indirectly, to provide a single chain polypeptide that may optionally be ultimately produced as a dimer in which the single chain polypeptides are coupled through inter-chain disulfide bonds formed within the Fc region. The nature of the fusing region is not critical.

The fusion may be direct between the two components, with the SIRPa component constituting the N-terminal end of the fusion and the Fc component constituting the C-terminal end.

Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, Clal, EcoRI, HindIII, Pstl, Sall and Xhol site and the like.

The linker amino acids typically and desirably have some flexibility to allow the Fc and the SIRPa components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G4S sequence (Gly-Gly-Gly-Gly-Ser [SEQ ID NO: 9]) which may repeat as (G4S)n (SEQ ID NO: 21) where n is 1, 2, 3 or more, or is based on (Gly)n, (Ser)n, (Ser-Gly)n or (Gly-Ser)n and the like. In another embodiment, the linker is GTELSVRAKPS [SEQ ID NO: 10]. This sequence constitutes SIRPa sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The term “CD47+” (or CD47+) is used with reference to the phenotype of cells targeted for binding by the present polypeptides. Cells that are CD47⁺ can be identified by flow cytometry using CD47 antibody as the affinity ligand. CD47 antibodies that are labeled appropriately are available commercially for this use (for example, the antibody product of clone B6H12 is available from Santa Cruz Biotechnology). The cells examined for CD47 phenotype can include standard tumour biopsy samples including particularly blood samples taken from the subject suspected of harbouring endogenous CD47⁺ cancer cells. CD47 disease cells of particular interest as targets for therapy with the present fusion proteins are those that “over-express” CD47. These CD47⁺ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

A SIRPaFc fusion protein provided herein can be administered in various dosage amounts within the range from about 0.0001 to 100 mg/kg.

In some embodiments, TTI-621 (SEQ ID NO: 7) is administered in the range of 0.01 to 30 mg/kg subject body weight. For example, TTI-621 dosages can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10.0 mg/kg. TTI-621 dosages can also include, for example 0.2-2 mg/kg, 0.7-2 mg/kg, 1-5 mg/kg, 2-5 mg/kg, or 2-10 mg/kg. These dosages of TTI-621 can be administered to a subject, for example, once a week (Q1W), once every two weeks (Q2W), once every three weeks (Q3W), once every four weeks (Q4W), two times a month, once a month, once every two months, or once every three months.

In some embodiments, TTI-622 (SEQ ID NO: 8) is administered in the range of 0.1 to 50 mg/kg subject body weight. For example, TTI-622 dosages can be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. TTI-622 dosages can also include, for example 2-40 mg/kg, 4-40 mg/kg, 5-50 mg/kg, 8-50 mg/kg, 8-40 mg/kg, 8-30 mg/kg, 8-28 mg/kg 10-50 mg/kg, 10-40 mg/kg, 10-30 mg/kg, 10-25 or 10-20 mg/kg. These dosages of TTI-622 can be administered to a subject, for example, once a week (Q1W), once every two weeks (Q2W), once every three weeks (Q3W), once every four weeks (Q4W), two times a month, once a month, once every two months, or once every three months.

In some embodiments, a SIRPaFc fusion protein provided herein [e.g., TTI-622 (SEQ ID NO: 8)] is administered as a “flat” (also referred to as a “fixed”) dose—i.e. the dose is the amount per patient, and the dose does not depend on the mass of the patient. In some embodiments, a SIRPaFc fusion protein such as TTI-622 is administered at a fixed dose of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, or 3600 mg. A fixed dose of SIRPaFc fusion protein may be administered in various regimens. In some embodiments, the dose is administered to a patient weekly (QW), every 2 weeks (Q2W), every 3 weeks (Q3W), or every 4 weeks (Q4W).

In some embodiments, a SIRPaFc fusion protein is administered at a dose between a) a lower level of 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, or 2200 mg and b) an upper level of 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, or 3600 mg, wherein the lower level is a smaller value than the upper level.

SIRPaFc proteins provided herein display negligible binding to red blood cells. There is accordingly no need to account for an RBC “sink” when dosing with SIRPaFc fusion proteins provided herein. Relative to other CD47 blockade drugs that are bound by RBCs, it is estimated that the present SIRPaFc fusions can be effective at doses that are less than half the doses required for drugs that become RBC-bound, such as CD47 antibodies. Moreover, the SIRPaFc fusion proteins provided herein are a dedicated antagonist of the SIRPa-mediated signal, they display negligible CD47 agonism when binding thereto. There is accordingly no need, when establishing medically useful unit dosing regimens, to account for any stimulation induced by the drug.

### Anti-CD19 Agents

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. The term also encompasses naturally occurring variants of CD19, e.g., splice variants, allelic variants, and isoforms.

“MOR208” and “XmAb 5574” and “tafasitamab” are used as synonyms for the anti-CD19 antibody according to Table 1. Table 1 provides the amino acid sequences of MOR208/tafasitamab. The MOR208 antibody is described in U.S. patent application Ser. No. 12/377,251, which is incorporated by reference in its entirety.

The term “anti-CD19 antibody” or “an antibody that binds to CD19” refers to an antibody that is capable of binding CD19 with sufficient affinity such that the antibody is useful as a diagnostic and/or therapeutic agent in targeting CD19.

The use of anti-CD19 antibodies in non-specific B cell lymphomas is discussed in WO2007076950 (US2007154473), which are both incorporated by reference in their entirety. The use of a CD19-antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (US U.S. Pat. No. 8,097,703) (Medarex); WO2008022152 (U.S. Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (U.S. Ser. No. 12/710,442) (Glenmark Pharmaceuticals), WO2012010562 and WO2012010561 (International Drug Development), WO2011147834 (Roche Glycart), and WO2012156455 (Sanofi), which are all incorporated by reference in their entireties.

In some embodiments, an anti-CD19 antibody used with methods and compositions provided herein is tafasitamab (MOR208). Tafasitamab has the sequences shown in Table 1 below.

TABLE 1
Tafasitamab sequences

| Description | SEQ ID NO: | Amino Acids |
|---|---|---|
| HCDR1 | SEQ ID NO: 11 | SYVMH |
| HCDR2 | SEQ ID NO: 12 | NPYNDG |
| HCDR3 | SEQ ID NO: 13 | GTYYYGTRVFDY |
| LCDR1 | SEQ ID NO: 14 | RSSKSLQNVNGNTYLY |
| LCDR2 | SEQ ID NO: 15 | RMSNLNS |
| LCDR3 | SEQ ID NO: 16 | MQHLEYPIT |
| VH | SEQ ID NO: 17 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSS |
| VL | SEQ ID NO: 18 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Full Heavy | SEQ ID NO: 19 | EVQLVESGGGLVKPGGSLKLSCAASGYTFTSY |
| chain | | VMHWRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light | SEQ ID NO: | DIVMTQSPATLSLSPGERATLSCRSSKSL |
| chain | 20 | QNVNGNTYLYWFQQKPGQSPQLLIYRMS |
| | | NLNSGVPDRFSGSGSGTEFTLTISSLEPE |
| | | DFAVYYCMQHLEYPITFGAGTKLEIKRTVA |
| | | APSVFIFPPSDEQLKSGTASVVCLLNNFYP |
| | | REAKVQWKVDNALQSGNSQESVTEQDSK |
| | | DSTYSLSSTLTLSKADYEKHKVYACEVTH |
| | | QGLSSPVTKSFNRGEC |

### E3 Ubigiuitin Ligiase Modulators

In some embodiments, combination therapies provided herein include an E3 ubiquitin ligase modulator. These modulators may also be referred to as immunomodulatory drugs (IMiDs). These modulators target the E3 ubiquitin ligase CUL4-RBX1-DDB1-CRBN (as known as CRL4^{CRBN}) and promote ubiquitination of the transcription factors IKZF1 and IKZF3 by CRL4^{CRBN}. Modulators of CRL4^{CRBN} include, for example, thalidomide, lenalidomide, and pomalidomide. They modulate CRL4 activity via binding to the CRBN subunit.

Dosing regimens and methods provided herein may be is useful to treat a variety of cancer cells. These include particularly 0047 positive and/or 0019 positive cancer cells, including liquid (hematological) and solid tumours. Solid tumours can be treated with the dosing regimens and methods provided herein, to reduce the size, number, or growth rate thereof and to control growth of cancer stem cells. Such solid tumours include 0047 positive and/or 0019 positive tumours in bladder, brain, breast, lung, colon, ovary, prostate, liver, and other tissues as well. In one embodiment, dosing regimens and methods provided herein can used to inhibit the growth or proliferation of hematological cancers. As used herein, “hematological cancer” refers to a cancer of the blood, and includes leukemia, lymphoma, and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma. In particular embodiments, dosing regimens and methods provided herein are useful to treat T cell lymphomas (TCL) that are a very heterogeneous group of lymphoid malignancies divided into cutaneous and peripheral TCL, which themselves are divided into nodal or extranodal types. CTCL derive from skin-homing T cells and consist of mycosis fungoides, Sezary syndrome, primary cutaneous T cell lymphoproliferative disorders, and anaplastic large cell lymphoma. The common features of TCL are aggressive course and poor response to therapy, with the exception of ALK and ALCL.

In some other embodiments, the hematological cancer treated with dosing regimens and methods is a CD47 positive or CD19 positive leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with a dosing regimen or method provided herein is a CD47 positive or CD19 positive lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

A SIRPaFc fusion protein or anti-CD19 antibody provided herein can be administered to the subject through any of the routes established for protein delivery, in particular intravenous, intradermal, and subcutaneous injection or infusion, or by oral or nasal administration.

Pharmaceutical compositions of a SIRPaFc fusion proteins or an anti-CD19 antibody may include dosage forms for intravenous, subcutaneous, intracutaneous, and intramuscular injections. These injectable preparations may be prepared by known methods. For example, the injectable preparations may be prepared, e.g., by dissolving, suspending, or emulsifying a fusion protein or antibody described above or its salt in a sterile aqueous medium or an oily medium conventionally used for injections. Exemplary pharmaceutical compositions comprising an antibody specific for CD19 that can be used in the context of the present disclosure are disclosed, e.g., in WO2008/022152 or WO2018/002031 which are both incorporated by reference in their entireties.

Incorporated by reference herein for all purposes is the content of U.S. Provisional Patent Application No. 63/351,117, filed Jun. 10, 2022, and U.S. Provisional Patent Application No. 63/494,500, filed Apr. 6, 2023.

The following examples of specific aspects for carrying out the present invention are offered for illustrative purposes only, and are not intended to limit the scope of the present invention in any way

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

In order that this invention may be better understood, the following examples are set forth. These examples are for purposes of illustration only and are not to be construed as limiting the scope of the invention in any manner.

### Example 1: Clinical Study of TTI-622 with Tafasitamab and Lenalidomide in Patients with Diffuse Large B Cell Lymphoma

The objective of this study is to evaluate the safety and preliminary efficacy of TTI-622 in combination with tafasitamab and lenalidomide in patients with relapsed/refractory (R/R) diffuse large B cell lymphoma (DLBCL) not eligible for stem cell transplantation.

This is a multicenter, international, 2 component (Phase 1b followed by Phase 2), study of TTI-622 in combination with tafasitamab and lenalidomide in participants with RR DLBCL who have completed at least 1 line of systemic treatment (at least 1 containing an anti-CD20 therapy), who are not candidates for autologous stem-cell transplantation (ASCT) or are not eligible for intensive chemotherapy, due to age (≥70 years old) or co-morbidities, or impossibility or unwilling to undergo ASCT. In part 2 of the study participants may not have received more than 2 lines of prior treatments (induction, transplant and consolidation will be considered one line of treatment). Prior treatment with CAR-T is allowed. In part 2 no more than 5 patients with prior CAR-T will be allowed on study.

Part 1 will be conducted in approximately 20 participants. The objectives of the Phase 1b component, which will precede the dose optimization portion of the study (part 2), is to evaluate the safety and tolerability, PK, PD of TTI-622 in combination with standard doses of tafasitamab and lenalidomide.

An escalation/de-escalation approach guided by mTPI-2 will be used in Phase 1b to identify the safe doses of TTI-622 when administered in combination with tafasitamab and lenalidomide as per Summary of Product Characteristics (SmPC) for tafasitamab and lenalidomide. The TTI-622 dose levels to be explored are as follows:
Cohort 1 (DL1): 4 mg/kg or 300 mg fixed dose TTI-622 QW C1-3 then C4+ Q2W
Cohort 2 (DL2): 10 mg/kg or 750 mg fixed dose TTI-622 QW C1-3 then C4+ Q2W
Cohort 3 (DL3): 18 mg/kg or 1350 mg fixed dose TTI-622 QW C1-3 then C4+ Q2W

Treatments will be administered in 28 day cycles. Lenalidomide will be administered up to cycle 12 while TTI-622 and tafasitamab will be administered until disease progression or intolerable toxicity.

For cycle 1, TTI-622 will be administered by IV infusion on days 1, 8, 15, and 22 in doses of 4 mg/kg, 10 mg/kg, 18 mg/kg, or fixed doses of 300 mg, 750 mg, or 1350 mg. Tafasitamab will be administered by IV infusion on days 1, 4, 8, 15, and 22 at 12 mg/kg. Lenalidomide will be administered orally on days 1-21 at 25 mg.

For cycle 2, TTI-622 will be administered by IV infusion on days 1, 8, 15, and 22 in doses of 4 mg/kg, 10 mg/kg, or 18 mg/kg, or fixed doses of 300 mg, 750 mg, or 1350 mg. Tafasitamab will be administered by IV infusion on days 1, 8, 15, and 22 at 12 mg/kg. Lenalidomide will be administered orally on days 1-21 at 25 mg.

For cycle 3, TTI-622 will be administered by IV infusion on days 1, 8, 15, and 22 in doses of 4 mg/kg, 10 mg/kg, or 18 mg/kg, or fixed doses of 300 mg, 750 mg, or 1350 mg. Tafasitamab will be administered by IV infusion on days 1, 8, 15, and 22 at 12 mg/kg. Lenalidomide will be administered orally on days 1-21 at 25 mg.

For cycles 4-12, TTI-622 will be administered by IV infusion on days 1, and 15 in doses of 4 mg/kg, 10 mg/kg, or 18 mg/kg, or fixed doses of 300 mg, 750 mg, or 1350 mg. Tafasitamab will be administered by IV infusion on days 1 and 15 at 12 mg/kg. Lenalidomide will be administered orally on days 1-21 at 25 mg.

For cycles 13 and onward, TTI-622 will be administered by IV infusion on days 1, and 15 in doses of 4 mg/kg, 10 mg/kg, or 18 mg/kg, or fixed doses of 300 mg, 750 mg, or 1350 mg. Tafasitamab will be administered by IV infusion on days 1 and 15 at 12 mg/kg.

Dosing regimens are summarized in Table 2 below:

TABLE 2
Study Intervention(s)

| Intervention Name | TTI-622 | Tafasitamab | Lenalidomide |
|---|---|---|---|
| Arm Name (group | Administered to all | Administered to all | Administered to all |
| of participants | participants | participants | participants |
| receiving a | | | |
| specific treatment | | | |
| or no treatment) | | | |
| Unit Dose | Cohort 1: 4 mg/kg or | 12 mg/kg | 25 mg |
| Strength(s) | 300 mg | | |
| | Cohort 2: 10 mg/kg or | | |
| | 750 mg | | |
| | Cohort 3: 18 mg/kg or | | |
| | 1350 mg | | |
| Route of | IV infusion | IV infusion | PO |
| Administration | | | |
| Administration Days | C1-3: days 1, 8, 15, 22 | C1: Days 1, 4, 8, | Days 1-21 of each |
| | C4+: days 1, 15 | 15 and 22 | cycle |
| | | C2-3: Days 1, 8, 15 | |
| | | and 22 | |
| | | C4+: Day 1 and 15 | |
| Duration of | Till disease | Till disease | Up to 12 cycles |
| Administration | progression | progression | |
| Use | Experimental | Experimental when | Experimental when |
| | | combined to TTI-622 | combined to TTI-622 |
| IMP or NIMP/AxMP | IMP | IMP | IMP |

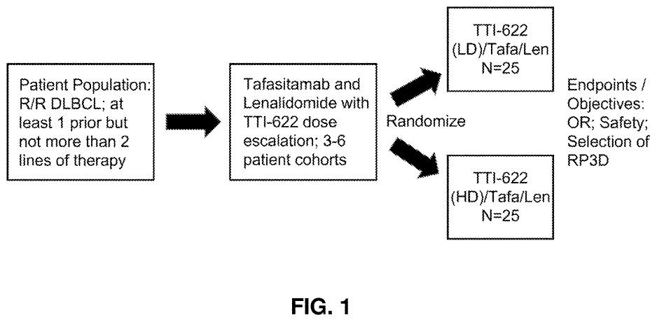

FIG. 1 provides a schematic of the study.

Sponsor may further escalate the dose based on emerging PK, PD, efficacy, and safety data. A safety review team, composed by sponsor personnel, including at least one medically qualified member, together with the investigators with participants enrolled in the specific cohort will review all safety data after each participant has been followed for at least 28 days from the first dose of the combination therapy. Patients will be considered evaluable for DLTs if they receive all doses as applicable (see above) within the DLT observation period or if they experience an adverse event (AE) meeting DLT criteria during that time. If a patient experiences a non-DLT event that requires a dose held per dose-modification guideline additional patients may be enrolled. If DL3 is not tolerated an additional cohort (DL4) may be explored where 18 mg/kg TTI-622 will be administered Q2W starting on C1 (or another dose level that is lower than DL3 but higher than DL2).

A minimum of 3 DLT-evaluable participants will be treated at each dose level of combination therapy. At least 9 DLT-evaluable participants will be treated at the estimated MTD or the highest tested dose level for TTI-622 before proceeding to Phase 2.

For Phase 2, the same or similar dosing regimen as used in Phase 1 described above may be used. Optionally, for Phase 2, the TTI-622 dose may be modified as compared to a Phase 1 regimen such that the TTI-622 is administered at a fixed dose of 300 mg, 600 mg, 900 mg, 1200 mg, 1500 mg, 1800 mg, 2100 mg, or 2400 mg, instead of a weight-based (mg/kg) dose. Optionally, when administered at a fixed dose, TTI-622 may be administered at the same frequency as provided in Phase 1 for a weight-based dose.

Participants must meet the following key inclusion criteria to be eligible for enrollment into the study:

Age and Sex: Properly consented male or female at least 18 years of age (or the minimum age of consent in accordance with local regulations) willing and able to comply with all study visits and assessments.

Disease Characteristics: Histologically confirmed progressing measurable relapsed and/or refractory CD19+ diffuse large B cell lymphoma (not otherwise specified); T cell/histiocyte rich large B-cell lymphoma, Epstein-Barr virus positive DLBCL, grade 3b follicular lymphoma or composite lymphoma with a DLBCL component with a subsequent DLBCL relapse, histological transformation from an earlier diagnosis of low-grade lymphoma into DLBCL (earlier diagnosis of follicular lymphoma, marginal zone lymphoma, chronic lymphocytic leukemia). Progression after transformation to DLBCL must be observed prior to enrollment.

At least 1 systemic treatment, not candidate per investigator or unwilling to undergo high dose chemotherapy or subsequent autologous stem cell transplant. At least 1 prior line must include an anti-CD20 containing treatment. In part 2 no more than 2 prior line of systemic treatment are allowed.

Participants with any of the following characteristics/conditions will be excluded:

### Medical Conditions:

1. Primary refractory DLBCL (response less than PR, or progression during or within 6 months after completion of first line of therapy).

2. Double or triple hit DLBCL and CNS lymphoma. (Double/triple hit: simultaneous detection of MYC with BCL2 and/or BCL6 translocation(s) defined by fluorescence in situ hybridization. MYC, BCL2, BCL6 testing prior to study enrolment is not required.)

3. Any history of malignancies other than DLBCL except for: Basal cell or squamous cell carcinoma of the skin or Bowen's disease that has adequately been treated; Prior follicular lymphoma transformed to DLBCL; or Any prior malignancies ≥3 years before randomization with no subsequent evidence of recurrence or progression without treatment regardless of the stage, or in situ, stage 0-1 cancer <3 years before randomization that has a remote probability of recurrence or progression per investigator.

4. Prior cancer treatment, radiotherapy within 2 weeks or major surgery within 2 weeks or unresolved grade >1 toxicities per NCI CTCAE v5.0 (except for alopecia).

5. Prior allogeneic stem cell transplantation; autologous stem cell transplantation within 12 weeks prior to enrolment, active GVHD (other than grade 1 skin involvement) or GVHD requiring treatment.

6. Significant cardiovascular disease, such as symptomatic congestive heart failure (New York Heart Association Class Ill or IV), symptomatic coronary artery disease, myocardial infarction, uncontrolled/unstable arrhythmia with treatment, unstable angina within the last 6 months, or prolonged QTcF interval >470 msec at screening. LVEF <40% as determined by a MUGA scan or ECHO. History of deep venous thrombosis or pulmonary embolism, threatening thromboembolism, or known thrombophilia. Participant must be willing to take prophylactic treatment for thromboembolic events).

7. Active hepatitis B or C or known HIV or COVID-19 infection (symptomatic or asymptomatic) or any infection requiring systemic therapy:

### Prior/Concomitant Therapy:

1. Prior treatment with anti-CD47 or anti-CD19 (other than CAR-T) or immunomodulatory agents like thalidomide or lenalidomide.

2. Live vaccines within 4 weeks prior to randomization and while on trial is prohibited except for administration of inactivated vaccines.

3. Use of corticosteroids except for the following: Intranasal, inhaled, eye drops, topical steroids, or local steroid injection (e.g., intra articular injection); Systemic corticosteroids at physiologic doses <10 mg/day of prednisone or equivalent.

The primary endpoint in Phase 1b is DLTs during the DLT observation period. Analyses of DLTs are based on the DLT Evaluable Set. The occurrence of DLTs and AEs constituting DLTs during the DLT observation period will be summarized and listed by dose level for participants enrolled in Phase 1b.

The primary endpoint in Phase 2 is OR per Lugano Response Classification Criteria 2014 as assessed by the investigator. Participants who do not have a post-baseline tumor assessment due to early progression of disease, who receive anti-cancer therapies other than the study interventions prior to reaching a CR or PR, or who die, have documented PD, or stop tumor assessments for any reason prior to reaching a CR or PR will be counted as non-responders in the assessment of ORR. ORR will be calculated along with the 2-sided 95% Cl using the Clopper-Pearson method.

Exemplary dosing regimens for Phase 2 and beyond include the following:

### Regimen: 600 mg TTI-622:

Treatments will be administered in 28 day cycles. Lenalidomide will be administered up to cycle 12 while TTI-622 and tafasitamab will be administered until disease progression or intolerable toxicity.

For cycle 1, TTI-622 will be administered by IV infusion on days 1, 8, 15, and 22 in fixed doses of 600 mg. Tafasitamab will be administered by IV infusion on days 1, 4, 8, 15, and 22 at 12 mg/kg. Lenalidomide will be administered orally on days 1-21 at 25 mg.

For cycle 2, TTI-622 will be administered by IV infusion on days 1, 8, 15, and 22 in fixed doses of 600 mg. Tafasitamab will be administered by IV infusion on days 1, 8, 15, and 22 at 12 mg/kg. Lenalidomide will be administered orally on days 1-21 at 25 mg.

For cycle 3, TTI-622 will be administered by IV infusion on days 1, 8, 15, and 22 in fixed doses of 600 mg. Tafasitamab will be administered by IV infusion on days 1, 8, 15, and 22 at 12 mg/kg. Lenalidomide will be administered orally on days 1-21 at 25 mg.

For cycles 4-12, TTI-622 will be administered by IV infusion on days 1 and 15 in fixed doses of 600 mg. Tafasitamab will be administered by IV infusion on days 1 and 15 at 12 mg/kg. Lenalidomide will be administered orally on days 1-21 at 25 mg.

For cycles 13 and onward, TTI-622 will be administered by IV infusion on days 1 and 15 in fixed doses of 600 mg. Tafasitamab will be administered by IV infusion on days 1 and 15 at 12 mg/kg.

### Regimen: 1200 mg TTI-622:

Treatments will be administered in 28 day cycles. Lenalidomide will be administered up to cycle 12 while TTI-622 and tafasitamab will be administered until disease progression or intolerable toxicity.

For cycle 1, TTI-622 will be administered by IV infusion on days 1, 8, 15, and 22 in fixed doses of 1200 mg. Tafasitamab will be administered by IV infusion on days 1, 4, 8, 15, and 22 at 12 mg/kg. Lenalidomide will be administered orally on days 1-21 at 25 mg.

For cycle 2, TTI-622 will be administered by IV infusion on days 1, 8, 15, and 22 in fixed doses of 1200 mg. Tafasitamab will be administered by IV infusion on days 1, 8, 15, and 22 at 12 mg/kg. Lenalidomide will be administered orally on days 1-21 at 25 mg.

For cycle 3, TTI-622 will be administered by IV infusion on days 1, 8, 15, and 22 in fixed doses of 1200 mg. Tafasitamab will be administered by IV infusion on days 1, 8, 15, and 22 at 12 mg/kg. Lenalidomide will be administered orally on days 1-21 at 25 mg.

For cycles 4-12, TTI-622 will be administered by IV infusion on days 1 and 15 in fixed doses of 1200 mg. Tafasitamab will be administered by IV infusion on days 1 and 15 at 12 mg/kg. Lenalidomide will be administered orally on days 1-21 at 25 mg.

For cycles 13 and onward, TTI-622 will be administered by IV infusion on days 1 and 15 in fixed doses of 1200 mg. Tafasitamab will be administered by IV infusion on days 1 and 15 at 12 mg/kg.

### Regimen: 1800 mg TTI-622:

Treatments will be administered in 28 day cycles. Lenalidomide will be administered up to cycle 12 while TTI-622 and tafasitamab will be administered until disease progression or intolerable toxicity.

For cycle 1, TTI-622 will be administered by IV infusion on days 1, 8, 15, and 22 in fixed doses of 1800 mg. Tafasitamab will be administered by IV infusion on days 1, 4, 8, 15, and 22 at 12 mg/kg. Lenalidomide will be administered orally on days 1-21 at 25 mg.

For cycle 2, TTI-622 will be administered by IV infusion on days 1, 8, 15, and 22 in fixed doses of 1800 mg. Tafasitamab will be administered by IV infusion on days 1, 8, 15, and 22 at 12 mg/kg. Lenalidomide will be administered orally on days 1-21 at 25 mg.

For cycle 3, TTI-622 will be administered by IV infusion on days 1, 8, 15, and 22 in fixed doses of 1800 mg. Tafasitamab will be administered by IV infusion on days 1, 8, 15, and 22 at 12 mg/kg. Lenalidomide will be administered orally on days 1-21 at 25 mg.

For cycles 4-12, TTI-622 will be administered by IV infusion on days 1 and 15 in fixed doses of 1800 mg. Tafasitamab will be administered by IV infusion on days 1 and 15 at 12 mg/kg. Lenalidomide will be administered orally on days 1-21 at 25 mg.

For cycles 13 and onward, TTI-622 will be administered by IV infusion on days 1 and 15 in fixed doses of 1800 mg. Tafasitamab will be administered by IV infusion on days 1 and 15 at 12 mg/kg.

### Regimen: 2400 mg TTI-622:

Treatments will be administered in 28 day cycles. Lenalidomide will be administered up to cycle 12 while TTI-622 and tafasitamab will be administered until disease progression or intolerable toxicity.

For cycle 1, TTI-622 will be administered by IV infusion on days 1, 8, 15, and 22 in fixed doses of 2400 mg. Tafasitamab will be administered by IV infusion on days 1, 4, 8, 15, and 22 at 12 mg/kg. Lenalidomide will be administered orally on days 1-21 at 25 mg.

For cycle 2, TTI-622 will be administered by IV infusion on days 1, 8, 15, and 22 in fixed doses of 2400 mg. Tafasitamab will be administered by IV infusion on days 1, 8, 15, and 22 at 12 mg/kg. Lenalidomide will be administered orally on days 1-21 at 25 mg.

For cycle 3, TTI-622 will be administered by IV infusion on days 1, 8, 15, and 22 in fixed doses of 2400 mg. Tafasitamab will be administered by IV infusion on days 1, 8, 15, and 22 at 12 mg/kg. Lenalidomide will be administered orally on days 1-21 at 25 mg.

For cycles 4-12, TTI-622 will be administered by IV infusion on days 1 and 15 in fixed doses of 2400 mg. Tafasitamab will be administered by IV infusion on days 1 and 15 at 12 mg/kg. Lenalidomide will be administered orally on days 1-21 at 25 mg.

For cycles 13 and onward, TTI-622 will be administered by IV infusion on days 1 and 15 in fixed doses of 2400 mg. Tafasitamab will be administered by IV infusion on days 1 and 15 at 12 mg/kg.

Optionally, other doses of TTI-622 may be used in the regimens described immediately above, such as 300 mg, 750 mg, 900 mg, 1350 mg, 1500 mg, 2100 mg, or other TTI-622 fixed doses provided elsewhere herein.

1. A method of treating a cancer in a patient, comprising administering a combination of a SIRPaFc fusion protein and an anti-CD19 antibody to the patient.

2. The method of claim 1, wherein the method further comprises administering an E3 ubiquitin ligase modulator.

3. The method of claim 1, wherein one, two, or all three of: i) the SIRPaFc fusion protein is administered Q1W or Q2W; ii) the anti-CD19 antibody is administered Q1W or Q2W: iii) the E3 ubiquitin ligase modulator is administered daily.

4. A method of treating a cancer in a patient, the method comprising administering a combination therapy of SIRPaFc fusion protein, an anti-CD19 antibody, and an E3 ubiquitin ligase modulator to the patient for at least a first cycle, a second cycle, a third cycle, and a fourth cycle,
wherein each cycle is 28 days,
wherein in the first cycle the SIRPaFc fusion protein is administered on days 1, 8, 15, and 22, the anti-CD19 antibody is administered on days 1, 4, 8, 15, and 22, and the E3 ubiquitin ligase modulator is administered on days 1-21,
wherein in the second cycle the SIRPaFc fusion protein is administered on days 1, 8, 15, and 22, the anti-CD19 antibody is administered on days 1, 8, 15, and 22, and the E3 ubiquitin ligase modulator is administered on days 1-21,
wherein in the third cycle the SIRPaFc fusion protein is administered on days 1, 8, 15, and 22, the anti-CD19 antibody is administered on days 1, 8, 15, and 22, and the E3 ubiquitin ligase modulator is administered on days 1-21, and
wherein in the fourth cycle the SIRPaFc fusion protein is administered on days 1 and 15, the anti-CD19 antibody is administered on days 1 and 15, and the E3 ubiquitin ligase modulator is administered on days 1-21.

5. The method of claim 4, wherein the method is continued for at least a fifth cycle and up to a twelfth cycle, and in the fifth to the twelfth cycle the SIRPaFc fusion protein is administered on days 1 and 15, the anti-CD19 antibody is administered on days 1 and 15, and the E3 ubiquitin ligase modulator is administered on days 1-21.

6. The method of claim 5, wherein the method is continued for at least a thirteenth cycle, wherein in the thirteenth cycle the SIRPaFc fusion protein is administered on days 1 and 15, the anti-CD19 antibody is administered on days 1 and 15, and no E3 ubiquitin ligase modulator is administered.

7. The method of claim 1, wherein the SIRPaFc fusion protein is administered at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg.

8. The method of claim 1, wherein the anti-CD19 antibody is administered at a dose of 12 mg/kg.

9. The method of claim 1, wherein the E3 ubiquitin ligase modulator is administered at a dose of 25 mg.

10. A method of treating a cancer in a patient, the method comprising administering a combination therapy of SIRPaFc fusion protein, an anti-CD19 antibody, and an E3 ubiquitin ligase modulator to the patient for at least a first cycle, a second cycle, a third cycle, and a fourth cycle,
wherein each cycle is 28 days,
wherein in the first cycle the SIRPaFc fusion protein is administered on days 1, 8, 15, and 22 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1, 4, 8, 15, and 22 at a dose of 12 mg/kg, and the E3 ubiquitin ligase modulator is administered on days 1-21 at a dose of 25 mg,
wherein in the second cycle the SIRPaFc fusion protein is administered on days 1, 8, 15, and 22 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1, 8, 15, and 22 at a dose of 12 mg/kg, and the E3 ubiquitin ligase modulator is administered on days 1-21 at a dose of 25 mg,
wherein in the third cycle the SIRPaFc fusion protein is administered on days 1, 8, 15, and 22 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1, 8, 15, and 22 at a dose of 12 mg/kg, and the E3 ubiquitin ligase modulator is administered on days 1-21 at a dose of 25 mg, and
wherein in the fourth cycle the SIRPaFc fusion protein is administered on days 1 and 15 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and the E3 ubiquitin ligase modulator is administered on days 1-21 at a dose of 25 mg.

11. The method of claim 10, wherein the method is continued for at least a fifth cycle and up to a twelfth cycle, and in the fifth to the twelfth cycle the SIRPaFc fusion protein is administered on days 1 and 15 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and the E3 ubiquitin ligase modulator is administered on days 1-21 at a dose of 25 mg.

12. The method of claim 11, wherein the method is continued for at least a thirteenth cycle, wherein in the thirteenth cycle the SIRPaFc fusion protein is administered on days 1 and 15 at a dose of 4 mg/kg, 10 mg/kg, or 18 mg/kg or a fixed dose of 300 mg, 600 mg, 750 mg, 1200 mg, 1350 mg, 1500 mg, 1800 mg, 2100 mg, or 2400 mg, the anti-CD19 antibody is administered on days 1 and 15 at a dose of 12 mg/kg, and no E3 ubiquitin ligase modulator is administered.

13. The method of claim 1, wherein the SIRPaFc fusion protein comprises a SIRPa polypeptide comprising the amino acid sequence of SEQ ID NO: 1.

14. The method of claim 1, wherein the SIRPaFc fusion protein comprises a SIRPa polypeptide comprising the amino acid sequence of SEQ ID NO: 2.

15. The method of claim 1, wherein the SIRPaFc fusion protein comprises the amino acid sequence of SEQ ID NO: 8.

16. The method of claim 1, wherein the SIRPaFc fusion protein comprises a SIRPa polypeptide comprising the amino acid sequence of SEQ ID NO: 1 or a variant thereof having one, two, three, four, or five amino acid substitutions as compared the sequence of SEQ ID NO: 1.

17. The method of claim 1, wherein the anti-CD19 antibody comprises a heavy chain variable region comprising an HCDR1 region comprising the sequence SYVMH (SEQ ID NO: 11), an HCDR2 region comprising the sequence NPYNDG (SEQ ID NO: 12), and an HCDR3 region comprising the sequence GTYYYGTRVFDY (SEQ ID NO: 13) and a light chain variable region comprising the sequence LCDR1 region comprising the sequence RSSKSLQNVNGNTYLY (SEQ ID NO: 14), an LCDR2 region comprising the sequence RMSNLNS (SEQ ID NO: 15), and an LCDR3 region comprising the sequence MQHLEYPIT (SEQ ID NO: 16).

18. The method of claim 1, wherein the anti-CD19 antibody comprises a heavy chain variable region of
(SEQ ID NO: 17)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSS
and
a light chain variable region of
(SEQ ID NO: 18)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIK.

19. The method of claim 1, wherein the anti-CD19 antibody comprises an Fc domain comprising an amino acid substitution at position S239 and/or I332, wherein the numbering is according to the EU index as in Kabat.

20. The method of claim 19, wherein the anti-CD19 antibody comprises an Fc domain comprising an S239D amino acid substitution and an I332E amino acid substitution, wherein the numbering is according to the EU index as in Kabat.

21. The method of claim 1, wherein the anti-CD19 antibody comprises a heavy chain region of
(SEQ ID NO: 19)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPDVF
LFPPKPKDTLMISRTPEVTCVVVDVSHEDPEVQFNWYVDGVEVHNAKTK
PREEQFNSTFRVVSVLTVVHQDWLNGKEYKCKVSNKALPAPEEKTISKT
KGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYT
QKSLSLSPGK
and
a light chain region of
(SEQ ID NO: 20)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

22. The method of claim 1, wherein the E3 ubiquitin ligase modulator comprises lenalidomide, pomalidomide, or thalidomide.

23. The method of claim 1, wherein one or both of: i) the cancer is a CD19 positive cancer and ii) the cancer is a CD47 positive cancer.

24. The method of claim 1, wherein the cancer is a hematological malignancy or a solid tumor cancer.

25.-26. (canceled)

27. A kit comprising a SIRPaFc fusion protein or an anti-CD19 antibody and instructions for use according to the method of claim 1.
